(19) United States
(12) Patent Application Publication
(10) Pub. No.: US 2024/0285170 A1
(43) Pub. Date: Aug. 29, 2024

(54) AUDIO VISUAL DETECTION PLATFORM FOR PATIENT MONITORING

(71) Applicant: Atapir, Inc., San Rafael, CA (US)

(72) Inventor: Arthur Wallace, SanRafael, CA (US)

(21) Appl. No.: 18/586,138

(22) Filed: Feb. 23, 2024

Related U.S. Application Data
(60) Provisional application No. 63/448,184, filed on Feb. 24, 2023.

(51) Int. Cl. A61B 5/00; A61B 5/0205; G16H 10/60; G16H 40/67; G16H 50/20; G16H 50/30

(52) U.S. Cl. CPC A61B 5/0077; A61B 5/0205; A61B 5/7435; G16H 10/60; G16H 40/67; G16H 50/20; G16H 50/30

(57) ABSTRACT

Systems and methods include a computer-implemented method for determining a diagnosis based on audio-visual information from a monitored person. A subject is monitored using one or more video systems. Vital health information of the subject is determined based at least on the monitoring. At least one diagnosis of the subject is determined based at least on the determined vital health information.

### CROSS-REFERENCE TO RELATED APPLICATIONS

This application claims the benefit under 35 USC 119(e) to Provisional Application No. 63/448, 184, filed on Feb. 24, 2023, and is hereby incorporated by reference in its entirety.

### TECHNICAL FIELD

This disclosure relates to an audio-visual detection platform, e.g., for use in patient monitoring in medical applications.

### BACKGROUND

Patients in hospitals and nursing homes have critical events such as respiratory and cardiac arrest, stroke, septic shock, perfusion problems with limb ischemia, pain, or hypotension. If these problems are detected quickly, or the progression of events leading to the event can be identified and corrective action taken, patients can avoid serious morbidity and mortality.

### SUMMARY

The Audio-Visual Detection Monitor (AVD-M) is a platform for patient and home monitoring. AVD-M is a remote, non-contact, monitoring system utilizing electronic camera or cameras, microphone and microphone arrays, speakers, machine vision, and artificial intelligence to monitor patients including all age groups (adults, children, neonates) in hospitals, nursing homes, at home, psychiatry facilities, emergency rooms, intensive care or step-down units, operating rooms, or other locations (e.g., trucks, plane cockpits, and machinery). AVD-M monitoring may utilize multiple colors or wavelengths but not limited to red-green-blue (RGB), infrared (IR), depth, and or stereoscopic imaging. The monitoring system monitors single or multiple parameters which may include but is not limited to: heart rate, respiratory rate, respiratory tidal volume, oxygen saturation (SpO2), perfusion, systolic blood pressure, diastolic blood pressure, mean blood pressure, pain, delirium, mood, out of bed situations, patient call system, turning and positioning information. stroke, regional anesthesia block, obstructive breathing, vomiting, shivering, congestive heart failure (CHF), heart rate variability, predictive composite variables that predict clinical outcomes, signs of suicide risk/ideation, depression, cough, temperature, skin blood flow, shock, patients leaving against medical advice, and compartment syndrome. The AVD-M system can also provide logistical information about patients including presence in room, room availability, state of cleaning for future occupancy, sound, noise, light, and sleep disturbances in hospital contributing to delirium, and or security information. The AVD-M system can be used for communication between clinicians and patients, or as a nursing call system, or as communication between patients and their family or friends. The AVD-M system can be used to control equipment in the hospital room either by voice or gesture. The AVD-M system also may provide a platform to host other parameters developed by other groups or companies that utilize the AVD-M monitor platform and or sensors. AVD-M monitoring software may also be applied utilizing patient's home computer and either a built-in camera or additional web cam to provide information for remote, telemedicine visits. These remote telemedicine visits may utilize red-green-blue, or other colors, infrared, and or depth cameras to provide multiple parameters for telemedicine visits and monitoring. The AVD-M system may be used with non-human animals to establish vital signs, disability, or diagnostic information in farming, veterinary, or zoo applications.

The AVD-M monitor is designed to create, for example, a smart room that provides continuous or intermittent, remote, non-contact monitoring of vital signs and is able to detect issues with hypoxia, hypoventilation, hypotension, shock, stroke, falls, pain, depression, mood, behavioral issues, delirium, vomiting, and obstructive breathing. The use of monitoring can reduce the incidence of respiratory and cardiac arrest and other clinical deterioration that can lead to significant morbidity and mortality. The AVD-M monitor can be used in hospitals, nursing homes, home, psychiatric facilities, prisons, airports, waiting rooms, and during telemedicine visits. The AVD-M monitoring system can also monitor operators of trucks, buses, drivers, airplanes, and machinery.

In one embodiment, there is a camera array that includes at least a red, green, blue (RGB) camera, an infrared (IR) camera, and a depth camera. There is a microphone array and speaker. There is a display. In an alternative embodiment there are fewer cameras or cameras of different types such as cameras sensitive to other spectrum of light or stereoscopic cameras or a lidar array. In another alternative embodiment AVD-M monitoring is achieved with the camera on a patient's home computer and the software is run on the home computer, mobile devices, or on a cloud or remote computer. The software only model may use whatever camera is available at the distant location such as RGB, IR, depth, or separate web camera, or camera arrays.

Other features, aspects and potential advantages will be apparent from the accompanying description and figures.

### DESCRIPTION OF DRAWINGS

FIG. 1 is a block diagram showing an example of an architecture for an Audio-Visual Detection Monitor (AVD-M) system configured to monitor a subject and capture vital information of the subject.

FIGS. 2A and 2B show an example deployment of the system of FIG. 1.

FIGS. 3A-3C collectively show a table of parameters and how they are processed to determine a diagnosis of a subject.

FIG. 4 is a flow chart of an example method for executing an audio visual detection platform for patient monitoring.

FIG. 5 shows an example of a computing device and an example of a mobile computing device that can be used to implement the techniques described herein.

Like reference symbols in the various drawings indicate like elements.

### DETAILED DESCRIPTION

This disclosure relates to local or remote, non-contact, patient monitoring designed for patients in hospitals, nursing homes, at home, adults, infants, or children, who may have cardiovascular, respiratory, medical, psychiatric, neurologic, vascular, or other problems. Patients in hospitals and nursing homes commonly are prescribed medications such as opiates and sedatives that suppress respiration. These medications can lead to hypercapnia, hypoventilation, obstructive sleep apnea, respiratory depression, and ultimately, respiratory and or cardiac arrest. The Audio-Visual Detection Monitor (AVD-M) is designed to detect respiratory depression and reduce the incidence of respiratory and cardiac arrest. Patients in hospital have other complications including but not limited to stroke, hypotension, septic shock, falls, delirium, pain, nausea, vomiting, depression, mood disturbances, and behavioral issues. Complications in hospitalized patients are frequently associated with delays in identification of clinical deterioration secondary to infrequent or inadequate monitoring.

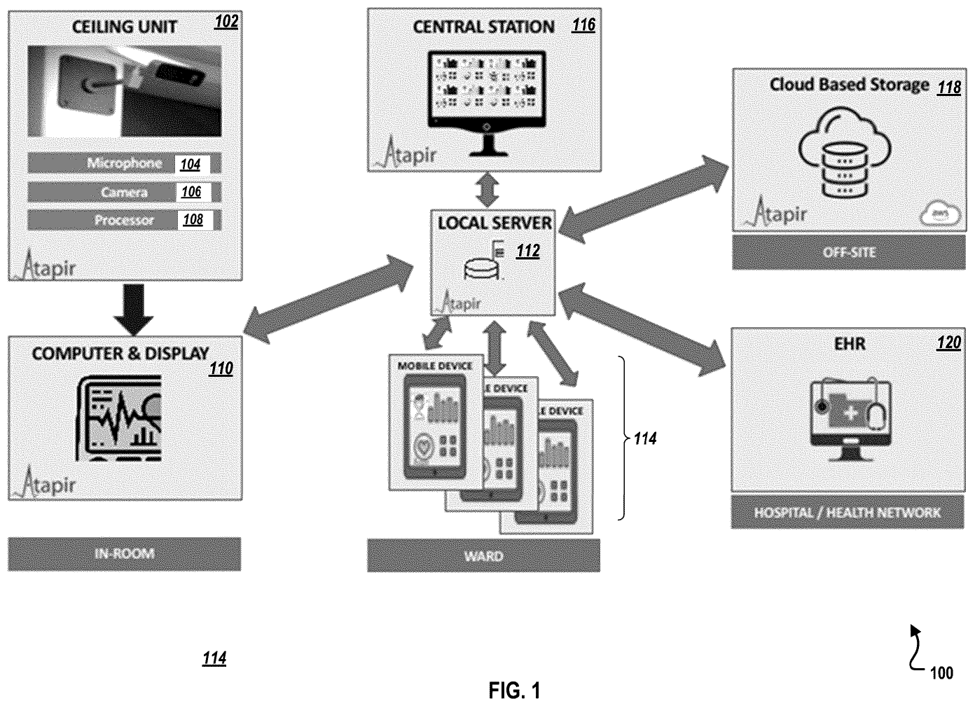

FIG. 1 is a block diagram showing an example of an architecture of an Audio-Visual Detection Monitor (AVD-M) system 100 configured to monitor a subject (e.g., a patient) and capture vital information of the subject. The system 100 serves as a platform that provides connectivity to a patient room display, one or more nursing stations, nurse displays, and the hospital network. The system allows integration with third-party algorithms and software, which can leverage camera hardware and connectivity capabilities. The system 100 includes a monitoring unit (e.g., a ceiling unit 102 or a wall unit) that includes one or more video systems, e.g., a microphone array 104 of one or more microphones, a camera array 106 of one or more cameras, and a processor 108 for processing information received from components of the ceiling unit 102.

A computer client 110 is configured to receive the vital information from the one or more video systems of the ceiling unit 102. The computer client 110 can determine, using at least the received vital information, at least one diagnosis of the subject. Information about the at least one diagnosis can be displayed with the vital information on one or more of a graphical user interface and using an audible user interface (e.g., using voice recognition software with an ear pod worn by the subject) of the computer client 110.

A server (e.g., implemented as one or more of a local server 112, a remote server, or the cloud) is in communication with the computer client 110 and peripheral systems (including the ceiling unit 102), including mobile devices 114, a central station 116, cloud-based storage 118, and an electronic health records (EHR) server 120. The central station is configured to: 1) receive and display the vital information and the at least one diagnosis from the computer client 110; 2) receive inputs from a user of the central station 116; and 3) send, responsive to the received inputs, commands to the computer client 110.

The mobile devices 114 can be configured to 1) receive the vital

information and the at least one diagnosis from the computer client 110; and 2) receive inputs from users of the mobile devices 114; and 3) send, responsive to the received inputs, commands to the computer client 110.

The cloud-based storage 118 can be configured to store, in the cloud, and provide access to, cloud-formatted subject data generated from the vital information and the at least one diagnosis generated. The EHR server 120 can be configured to store and provide access to EHR for the subject.

In some implementations, the vital information of the subject can include vital signs of a human being (e.g., a casualty, a victim, an athlete, a firefighter/first-responder, or a hostage) or one or more animals. The vital information can be collected from a remote location (e.g., a battlefield, a hostage situation, a crime scene, a disaster scenario, an emergency location, a sporting event, a security checkpoint, or a vehicle). In these and other examples, the one or more video systems can include at least one camera and at least one microphone mounted on one or more of a drone, a fixed location, or a robotic device. The computer client can be nearby or distant from the subject and can provide a display summarizing: i) the vital signs of the subject, and ii) at least one conclusion regarding a condition of the subject.

The system 100 can be implemented with an open architecture. Components of the system 100 can be used with application programming interfaces (APIs) that allows implementations of the system 100 to incorporate and monetize third-party solutions for increased functionality. As a software solution, as opposed to a hardware product, additional capabilities can be added as simple software upgrades without incurring high costs. For example, parameters such as pain, delirium, perfusion, stroke, and additional capabilities can be implemented incrementally as simple software upgrades without incurring high costs. Features can include measuring risk factors (e.g., associated with delirium) including light levels at night, sound levels at night, frequency of alarms, time-of-day of alarms, staff interactions with the patient, windows present in a room, and information from the electronic health records, including patient age, risk factors, medications, and surgery.

The system 100 can include digital cameras and a microphone array. The system can be implemented with software that employs artificial intelligence (AI), machine vision, and proprietary algorithms and software. The system 100 can provide connectivity to the hospital's information technology (IT) system(s) and smart alarms.

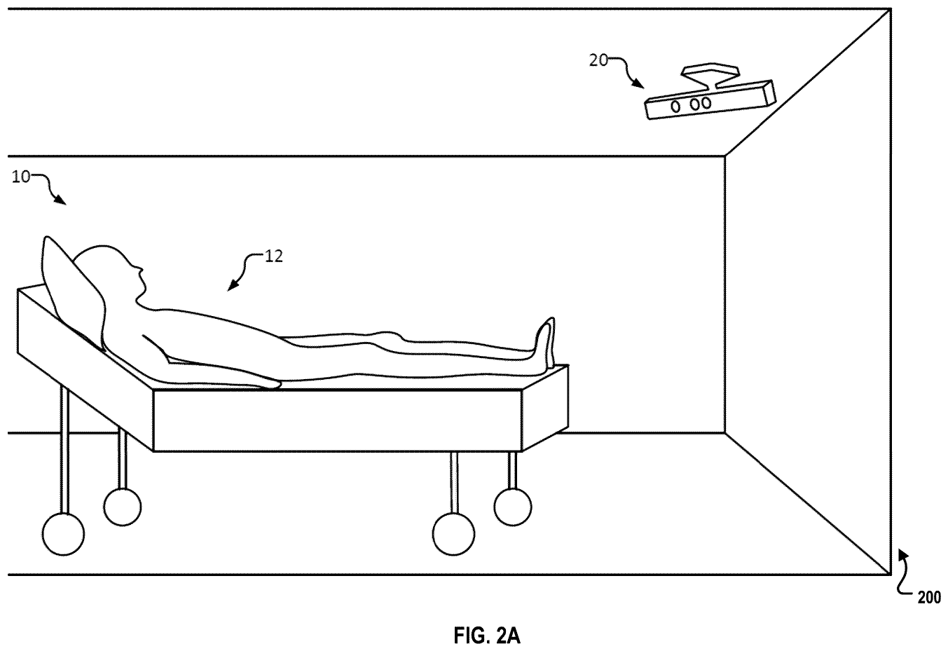

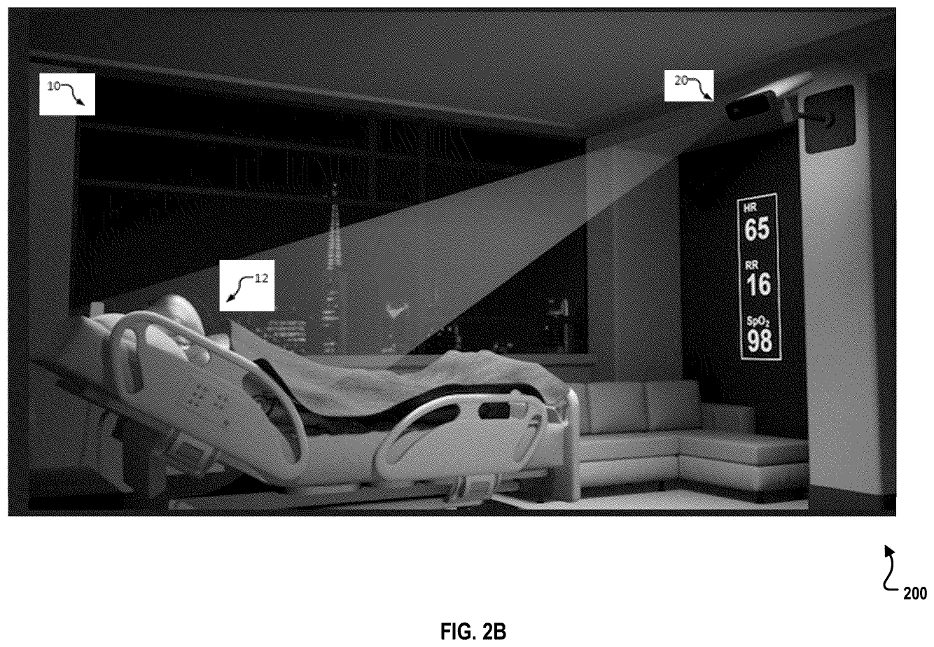

FIGS. 2A and 2B show an example deployment 200 of the system of FIG. 1. The deployment 200 includes a monitoring area 10 in which a patient 12 is positioned for view by an AVD-M device 20. The patient 12 is in a bed with a camera array observing the patient 12. A display on the wall displays vital signs of the patient.

In some implementations, AVD-M prototypes and devices can be verified and validated using other devices running in parallel, e.g., a pulse oximeter attached to a finger of a subject to check for accuracy. In these and other cases, the AVD-M can detect HR, RR, and SpO2 remotely.

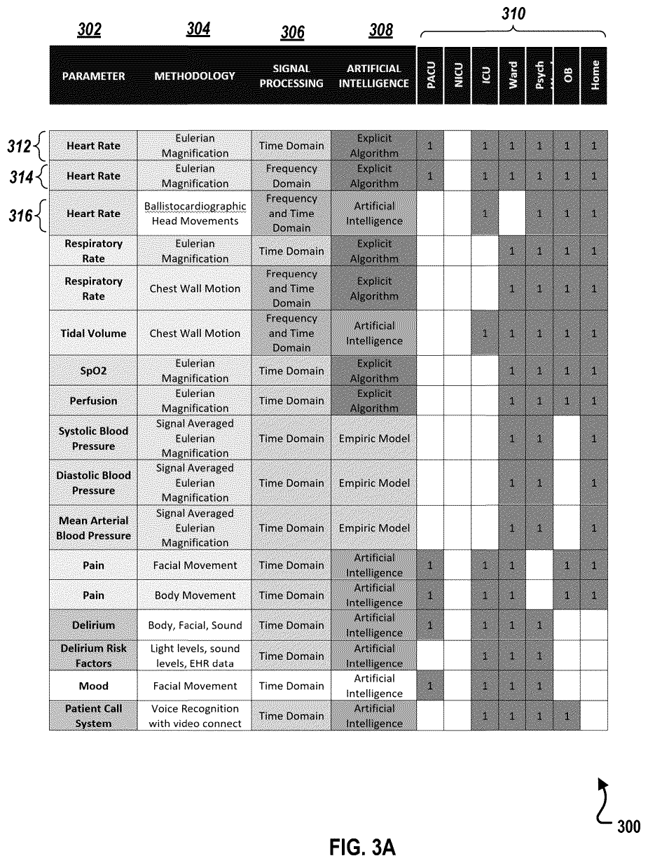

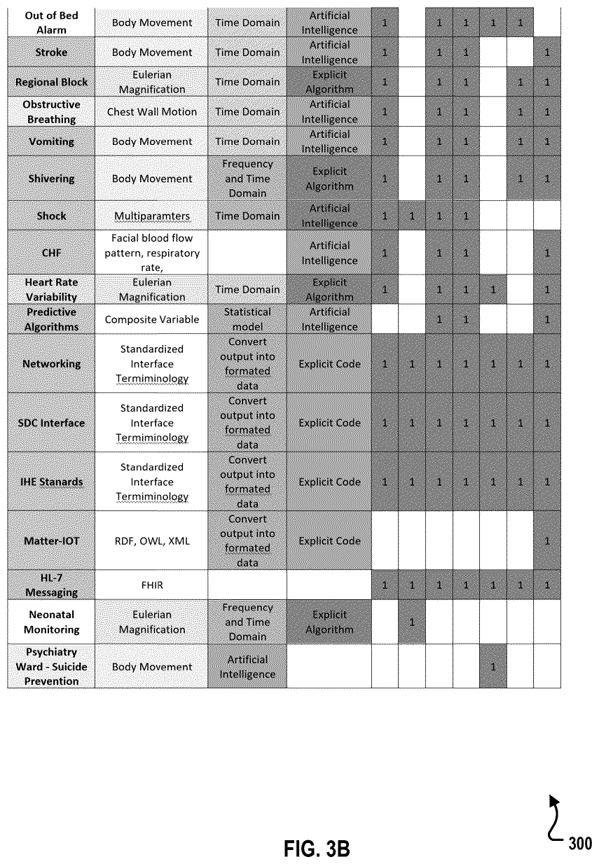

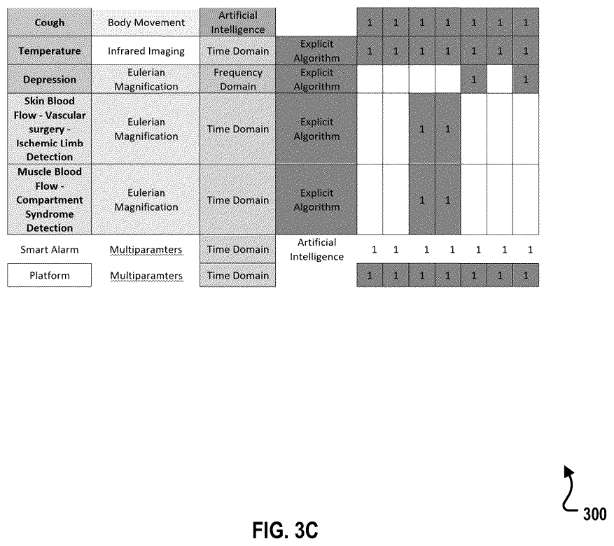

FIGS. 3A-3C collectively show a table 300 of parameters 302 and how they are processed to determine a diagnosis of a subject. For each parameter 302, one or more methodologies 304, signal processing 306, algorithms 308 (e.g., artificial intelligence) can be used to generate a diagnosis. Indicators 310 indicate which locations a given combination of parameter 302, methodology 304, signal processing 306, and algorithm 308 can be used. The locations can include, for example, post anesthesia care unit (PACU), neonatal NICU (NICU), ICU, ward, psych ward, observation (OB), and home. Example rows 312, 314, and 316 in the table 300 show that the heartrate parameter 302 can be analyzed using different combinations of methodologies 304 (e.g., Eulerian magnification and ballistocardiographic head movements), signal processing 306 (e.g., time domain, frequency domain, and combinations thereof), and algorithm 308 (e.g., artificial intelligence and explicit algorithms) to determine a diagnosis when used in applicable locations.

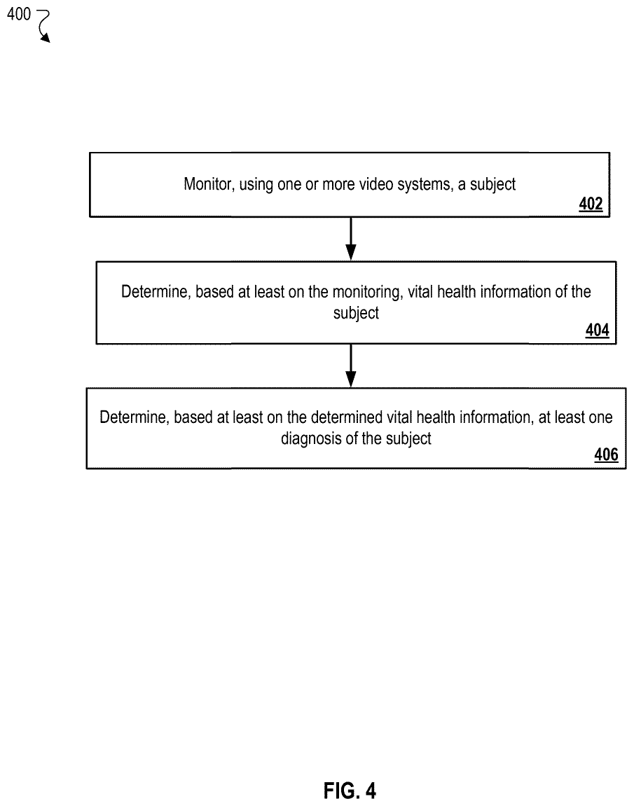

FIG. 4 is a flow chart of an example method 400 for executing steps of a patient monitoring system.

At 402, a subject is monitored using one or more video systems. For example, a patient in a hospital setting can be monitored by the ceiling unit 102 as described with reference to FIG. 1.

At 404, vital health information of the subject is determined based at least on the monitoring. As an example, the computer client 110 can determine the patient's vital information based on the monitoring.

At 406, at least one diagnosis of the subject is determined based at least on the determined vital health information. For example, types of diagnoses as listed in FIGS. 3A-3C can be determine by the computer client 110 using information from other components of the system 100 (including elements 102, 110, 112, 118, and 120), as described with reference to FIG. 1.

The AVD-M monitor is designed to remotely identify these issues through algorithms that utilize machine vision and artificial intelligence. There are a number of logistical issues in hospitals such as presence of patient in the room, room cleaning, turning of patients in beds, room availability and preparation for the next patient which can be identified through remote, non-contact, video monitoring utilizing machine vision and artificial intelligence. Patients having remote telemedicine visits or remote at home monitoring can have parameters monitored either intermittently, during or after a medical exam, or continuously utilizing their home computer and camera.

Patients in hospitals and nursing homes have critical events such as respiratory and cardiac arrest, stroke, septic shock, perfusion problems with limb ischemia, pain, and hypotension. If these problems are detected quickly or the progression of events leading to the event can be identified and corrective action taken, patients can avoid serious morbidity and mortality. Many patients in hospitals are not monitored continuously with ECG, pulse oximetry, continuous blood pressure, and capnography. Patients who are not continuously monitored have vital signs taken intermittently sometimes every 4-8 hours. Patients in nursing homes may have even more infrequent evaluation and vital sign measurements. When we review critical events leading to respiratory and cardiac arrest, frequently the clinical deterioration is a slow process over many hours. If this clinical deterioration can be identified early, it is easier to correct the problem and avoid the critical event that leads to cardiac and respiratory arrest.

When patients have a stroke in hospital, it is critical to detect the change in neurologic function rapidly. It is also critical to identify when the patient was last seen to be normal. Many reperfusion strategies for reducing the sequelae of strokes require the time the patient was last seen to be normal. If the patient went to bed at 9 pm and was not examined between 9 pm and 7 am when the stroke was identified, clinical teams will use the time the patient was last seen to be normal, 9 pm, in the algorithms to decide if reperfusion of the brain will be advantageous or clinically safe. The AVD-M system can continuously monitor patient movement. It can identify when strokes that affect motor or speech abnormalities occur and provide the time when the patient was last seen to move the affected area. This ability to continuously track patient movement will reduce the time to identify motor abnormalities but will also provide improved information on when the patient was last seen to have normal or baseline motor function. In some implementations, smart alarms can be provided to reduce false alarms with adequate sensitivity and specificity by combining alarms and validating alarms. In some implementations, logistical information can be generated (e.g., based on the monitoring in multiple rooms) and provided to hospital management including bed occupancy, bed availability, location of clinical staff, cleaning of rooms, patient turning, and security alerts.

Hospitals need to be able to assess logistical issues in a large number of rooms continuously and rapidly. The AVD-M system provides continuous monitoring of rooms and can provide information on occupancy, cleaning status, fall prevention, behavioral issues, and patient turning information.

Telemedicine visits are becoming the standard for multiple services including but not limited to internal medicine, family medicine, pediatrics, psychiatry, psychology, and substance abuse. The AVD-M system can be used with the patient's home computer and camera, either built into the computer or webcam. This information can supplement the telemedicine visit with multiple parameters including, but not limited to, heart rate, respiratory rate, SpO2, blood pressure, perfusion, breathing patterns, mood, and pain.

The AVD-M system provides multiple parameters including but not limited to vital signs, mood, pain, mobility, and patient status remotely without contact with the patient. AVD-M identifies problems that can lead to significant morbidity and mortality by continuously monitoring patients so that issues can be rapidly identified and addressed prior to adverse outcomes.

The current, non-AVD-M monitoring approaches rely on sensors applied to the patient including ECG leads, pulse oximetry sensors attached to patient's fingers, nose, ear, or other areas, blood pressure cuffs on the arm or leg, invasive arterial catheterization, and capnography with sensors in or near the patient's airway. These monitors require patient cooperation, time to place the monitor, maintenance of the sensor including replacement or repositioning when they disconnect. These monitors with sensors on the patient limit patient movement and make it difficult for patients to get out of bed because the cables, wires, and sensors tether the patient to the bed and monitor system. AVD-M monitoring can be used without sensors on the patient. Additionally, AVD-M monitoring provides information that traditional monitoring systems cannot provide such as stroke, perfusion, nausea, pain, mood, vomiting, out of bed alarms. AVD-M monitoring can be used in addition to traditional, sensor-based monitoring to provide these additional parameters. At present telemedicine visits require additional monitors to be provided to the patient including blood pressure cuffs, finger pulse oximeters. With AVD-M telemedicine these parameters can be monitored using the patient's computer and electronic camera.

AVD-M monitoring provides continuous and or intermittent monitoring without application of sensors to the patient. AVD-M monitoring can provide additional parameters that cannot be easily monitored with traditional sensors placed on the patients.

AVD-M monitoring provides continuous patient monitoring without sensors on the patient.

### Definitions

AVD-M Audiovisual detection monitor

Heart Rate—(HR) The rate of the beating of the heart in beats per minute.

Respiratory Rate—(RR) The rate of breathing in breaths per minute.

Tidal Volume—(TV) The volume of air exchanged on a single breath in milliliters per breath.

Pulse Oximetry (SpO₂) The oxygen saturation of arterial blood in percentage.

Systolic Blood Pressure (SBP) The systolic arterial blood pressure in mmHg.

Diastolic Blood Pressure (DBP) The diastolic blood pressure in mmHg. Mean Arterial Blood Pressure (MAP) The mean blood pressure in mmHg.

Pain—Measured by VAS (Visual Analogue Scale), a dimensionless, subjective measure of pain. Where zero (0) is defined as no pain and ten (10) is defined as the worst pain imaginable. Can also be measured by Critical-Care Pain Observation Tool (CPOT), the possible total score ranges from 0 (no pain) to 8 (maximum pain). The CPOT cutoff score was >2 during nociceptive procedures. A limitation of the CPOT is the lack of sufficient research in delirious critically ill patients. The CPOT includes evaluation of four different behaviors (facial expressions, body movements, muscle tension, and compliance with the ventilator for mechanically ventilated patients or vocalization for nonintubated patients) rated on a scale of zero to two with a total score ranging from 0 to 8. There are multiple other clinical measures of pain that can be used to calibrate the AVD-M pain detection system.

Delirium—Delirium is a serious change in mental abilities. It results in

confused thinking and a lack of awareness of someone's surroundings. The disorder usually comes on quickly within hours or a few days. Delirium can often be traced to one or more factors. There are multiple diagnostic algorithms based on four cardinal features of delirium: (1) acute onset and fluctuating course; (2) inattention; (3) disorganized thinking; and (4) altered level of consciousness. The CAM diagnostic algorithm evaluates four key features of delirium: 1) Acute Change in Mental Status with Fluctuating Course, 2) Inattention, 3) Disorganized Thinking, and 4) Altered Level of Consciousness. There are multiple scores for delirium. The Cognitive Test of Delirium (CTD) evaluates five items, each of which receives a score of 0, 2, 4, or 6 points. Thus, the scale has a total of 30 points possible. There is no subdivision described in the literature that correlates the severity levels with the respective CTD values. The Delirium Rating Scale (DRS)3 is a widely used delirium rating instrument that specifically, sensitively, and reliably measures delirium symptoms as rated by a psychiatrist or trained clinician. The Confusion Assessment Method-Intensive Care Unit (CAM-ICU) score is a validated and commonly used score to help monitor patients for the development or resolution of delirium. It is an adaptation of the CAM score for use in ICU patients.

Mood—The conception of mood in cognitive psychology is derived from the analysis of emotion. Mood is considered as a group of persisting feelings associated with evaluative and cognitive states which influence all the future evaluations, feelings and actions. Multiple parameters of mood will be presented including but not limited to anger, disgust, fear, happiness, sadness, surprise, and neutral.

Patient Call System—-Two-way video conferencing between patient and nurse, doctor, or family. Can be used to communicate needs, emergencies, and or requests. Also has speech recognition and text to speech capabilities with natural language processing and text messaging to staff and from staff or family to patient.

Out of Bed Alarm—System to track patient location in room and or in bed. If set, will send alarm if patient attempts to leave the bed or chair or designated location.

Stroke—A stroke occurs when something blocks blood supply to part of the brain or when a blood vessel in the brain bursts. In either case, parts of the brain become damaged or die. A stroke can cause lasting brain damage, long-term disability, or even death. The warning signs of stroke include: Numbness or weakness in the face, arm, or leg; Confusion or trouble speaking or understanding speech, Trouble seeing in one or both eyes; Trouble walking, dizziness, or problems with balance; severe headache with no known cause. The AVD-M system will track patient movement and speech. Changes in patient movement or speech will be evaluated as indication of possible strokes.

Regional Block—Regional anesthesia utilizes local anesthetic to anesthetize areas of the body. The types of regional anesthesia include spinal anesthesia (also called subarachnoid block), epidural anesthesia, and nerve blocks. When local anesthesia is used, motor movement of the affected area may be reduced depending on the location of the block and the concentration of the local anesthetic. Local anesthesia affects nerves based on their myelination and the size of the nerves. Sympathetic nerves, which control local vasodilation, are small fiber and blocked by local anesthesia. Vasodilation, caused by sympathetic blockade, can be detected by changes in skin blood flow, which can be visualized by changes in skin pulsatility signal. The dermatomal level of a regional block will be reported by changes in pulsatility of the skin in a dermatome. Dermatomes are areas of skin, each of which is connected to a single spinal nerve. Together, these areas create a surface map of the body.

Obstructive breathing—Obstruction of the inflow of the upper airway have the following signs. Agitation or fidgeting, bluish color to the skin (cyanosis), Changes in consciousness, Choking, Confusion, Difficulty breathing, gasping for air, leading to panic, Unconsciousness (lack of responsiveness), Wheezing, crowing, whistling, or other unusual breathing noises indicating breathing difficulty. The tongue is the most common cause of upper airway obstruction, a situation seen most often in patients who are comatose or who have suffered cardiopulmonary arrest. Other common causes of upper airway obstruction include edema of the oropharynx and larynx, trauma, foreign body, and infection. Discordant respiration or obstructive breathing can be identified by abnormal motion of the chest. Partial airway obstruction: breathing labored, gasping or noisy can be caused by some air escaping from the mouth. patient coughing or making a ‘crowing’ noise. Motion of the chest will be tracked by machine vision. The sounds of respiration will be tracked by the microphone and microphone array.

Vomiting—Vomiting (also known as emesis and throwing up) is the involuntary, forceful expulsion of the contents of one's stomach through the mouth and sometimes the nose. Vomiting will be identified by tracking motion of the abdomen, chest, neck, and head using machine vision.

Shivering—Shivering (also called shuddering) is a bodily function in response to cold and extreme fear in warm-blooded animals. When the core body temperature drops, the shivering reflex is triggered to maintain homeostasis. Skeletal muscles begin to shake in small movements, creating warmth by expending energy. Shivering can also be a response to fever, as a person may feel cold. During fever, the hypothalamic set point for temperature is raised. The increased set point causes the body temperature to rise (pyrexia), but also makes the patient feel cold until the new set point is reached. Severe chills with violent shivering are called rigors. Rigors occur because the patient's body is shivering in a physiological attempt to increase body temperature to the new set point. Shivering will be identified by machine vision.

Heart failure (HF), also known as congestive heart failure (CHF), is a syndrome, a group of signs and symptoms caused by an impairment of the heart's blood pumping function. Symptoms typically include shortness of breath, excessive fatigue, and leg swelling. The shortness of breath may occur with exertion or while lying down and may wake people up during the night. Chest pain, including angina, is not usually caused by heart failure, but may occur if the heart failure was caused by a heart attack. The severity of the heart failure is measured by the severity of symptoms during exercise. Other conditions that may have symptoms similar to heart failure include obesity, kidney failure, liver disease, anemia, and thyroid disease. Congestive heart failure will be identified by machine vision and artificial intelligence analysis utilizing multiple parameters.

Heart Rate Variability (HRV) Heart rate variability (HRV) is the physiological phenomenon of variation in the time interval between heartbeats. It is measured by the variation in the beat-to-beat interval. Other terms used include: “cycle length variability”, “R-R variability” (where R is a point corresponding to the peak of the QRS complex of the ECG wave; and RR is the interval between successive Rs), and “heart period variability”. Heart rate variability will be measured by the variation in the interval of peak signal from skin pulpability using machine vision.

Predictive Algorithms—Composite variables combining multiple parameters such as heart rate, respiratory rate, SpO₂, blood pressure, and temperature, can be combined together to predict the risk of impending clinical events. The combination of parameters will be used to create parameters that will predict the likelihood of clinical events so that clinical care can be provided in advance of severe deterioration interrupting the chain of events that can lead to respiratory and cardiac arrest.

Cough—A cough is a sudden expulsion of air through the large breathing passages which can help clear them of fluids, irritants, foreign particles, and microbes. As a protective reflex, coughing can be repetitive with the cough reflex following three phases: an inhalation, a forced exhalation against a closed glottis, and a violent release of air from the lungs following opening of the glottis, usually accompanied by a distinctive sound. Frequent coughing usually indicates the presence of a disease. Coughing will be detected by sound using the microphone and microphone array.

Temperature—Patient temperature is a standard vital sign measured either in degrees Fahrenheit or degrees Celsius. Normal human body-temperature (normothermia, euthermia) is the typical temperature range found in humans. The normal human body temperature range is typically stated as 36.5-37.5° C. (97.7-99.5° F.).

Depression is a mental state of low mood and aversion to activity. It affects more than 280 million people of all ages (about 3.5% of the global population). Classified medically as a mental and behavioral disorder, the experience of depression affects a person's thoughts, behavior, motivation, feelings, and sense of well-being. The core symptom of depression is said to be anhedonia, which refers to loss of interest or a loss of feeling of pleasure in certain activities that usually bring joy to people. Depressed mood is a symptom of some mood disorders such as major depressive disorder and dysthymia; it is a normal temporary reaction to life events, such as the loss of a loved one; and it is also a symptom of some physical diseases and a side effect of some drugs and medical treatments. It may feature sadness, difficulty in thinking and concentration and a significant increase or decrease in appetite and time spent sleeping. People experiencing depression may have feelings of dejection, hopelessness, and suicidal thoughts. It can either be short term or long term. Depression can be measured by patient responses to a PHQ-9 survey tool. The 9-question Patient Health Questionnaire (PHQ-9) is a diagnostic tool introduced in 2001 to screen adult patients in a primary care setting for the presence and severity of depression. It rates depression based on the self-administered Patient Health Questionnaire (PHQ). The PHQ-9 takes less than 3 minutes to complete and simply scores each of the 9 DSM-IV criteria for depression based on the mood module from the original PRIME-MD.

Skin Blood Flow—Hemodynamics are the dynamics of blood flow. The circulatory system is controlled by homeostatic mechanisms of autoregulation, just as hydraulic circuits are controlled by control systems. The hemodynamic response continuously monitors and adjusts to conditions in the body and its environment. Hemodynamics explains the physical laws that govern the flow of blood in the blood vessels. Blood flow ensures the transportation of nutrients, hormones, metabolic waste products, oxygen, and carbon dioxide throughout the body to maintain cell-level metabolism, the regulation of the pH, osmotic pressure and temperature of the whole body, and the protection from microbial and mechanical harm. Blood is a non-Newtonian fluid and is most efficiently studied using rheology rather than hydrodynamics. Because blood vessels are not rigid tubes, classic hydrodynamics and fluids mechanics based on the use of classical viscometers are not capable of explaining hemodynamics. Skin blood flow will be measured by pulsatile changes in the light reflection from the skin in visible and or infrared spectrum.

Muscle Blood Flow—Blood flow in the muscle underlying skin will be assessed by measurement of pulsatile changes in the light reflection from the skin and subcutaneous tissue in visible and or infrared spectrum.

Shock—Shock is the state of insufficient blood flow to the tissues of the body as a result of problems with the circulatory system. Initial symptoms of shock may include weakness, fast heart rate, fast breathing, sweating, anxiety, and increased thirst. This may be followed by confusion, unconsciousness, or cardiac arrest, as complications worsen. There are multiple types of shock including: vasodilatory shock, septic shock, cardiogenic shock, hypovolemic shock, anaphylactic shock, and neurogenic shock.

Networking—Signals from the AVD-M monitor will be transmitted to the hospital electronic health care record (EHR), centralized monitoring station, individual clinician computers, remote computers, remote hospital locations using standard networking capabilities including but not limited to JSON, HL7, FHIR, and or proprietary formats. Information will also be obtained from the electronic health record that may or may not be included as input parameters in predictive models or displayed.

Eulerian Video Magnification, takes a video sequence as input, separates the individual colors (R, G, B, I, other), and applies temporal filtering to the frames. The resulting signal is then amplified to reveal hidden information such as pulsatility from blood flow in the skin.

Platform—The AVD-M system will consist of either single or multiple electronic cameras with different light sensitivity. It may consist of red-green-blue (RGB) camera(s), infrared camera(s), stereoscopic camera(s), or depth camera(s). It may consist of multiple or singular cameras or a camera that is part of a personal computer, personal tablet computer, or additional camera (web camera). It will also consist of a computer or computers with or without a graphical processor unit (GPU) for array processing. It may or may not have light sources in visible or infrared light. It may have cameras with different light spectrum from standard RGB cameras. It may have wide angle, normal, or telephoto lens with or without autofocusing. It may or may not have a microphone and or microphone array. It may or may not have speakers to generate sound. It may or may not have a display, keyboard, mouse, and networking capabilities. It will have software that is either resident on the computer or hosted on other computers or on the cloud. Analysis of images may be local on the computer, on another local computer, or hosted on the cloud or other servers. The AVD-M system may or may not be able to run software from other companies that provide additional parameters as a platform for monitoring.

Vital signs (also known as vitals) are a group of the four to six most crucial medical signs that indicate the status of the body's vital (life-sustaining) functions. These measurements are taken to help assess the general physical health of a person, give clues to possible diseases, and show progress toward recovery. The normal ranges for a person's vital signs vary with age, weight, gender, and overall health. There are four primary vital signs: body temperature, blood pressure, pulse (heart rate), and breathing rate (respiratory rate), often notated as T, BP, HR, and RR. However, depending on the clinical setting, the vital signs may include other measurements called the “fifth vital sign” or “sixth vital sign”. Vital signs are recorded using the LOINC internationally accepted standard coding system. Early warning scores have been proposed that combine the individual values of vital signs into a single score. This was done in recognition that deteriorating vital signs often precede cardiac arrest and/or admission to the intensive care unit. Used appropriately, a rapid response team can assess and treat a deteriorating patient and prevent adverse outcomes.

The system described herein provides a method of measurement of the above parameters.

### Approach to Measurement

The generalized approach to measurement is to have a video signal from RGB or IR or Depth cameras or stereoscopic cameras or cameras of other frequencies. Different algorithms require different approaches to measurement. The camera system collects images and video. Algorithms on the computer identify the patient separate from the background image. The patient's face is identified using facial recognition algorithms such as but not limited to Haar Cascade based algorithms. The patient may or may not then be identified by facial recognition to ensure the correct person is monitored. Pulsatility in the skin is then assessed by Eulerian magnification algorithms or other techniques in one or multiple colors such as red, green, blue, and or infrared. A pulsatile signal from in the skin may be assessed in one or multiple colors (R, G, B, I, other). One embodiment utilizes the area above the eyebrows on the face for measurement of heart rate, SpO2, and respiratory rate. Signals from the chest or other areas may also be used for respiratory rate. Skin pulsatility imaging or motion of the chest utilizing depth imaging or stereoscopic imaging can be used to measure respiratory rate, tidal volume, and breathing patterns. Imaging can be achieved in the dark with infrared. If additional light is needed, lights of appropriate color and intensity can be turned on including but not limited to red, green, blue, infrared. Lights may or may not be targeted or diffuse. Lighting may be controlled automatically by the system when needed or on control of clinical staff or patient.

Heart Rate: Can be measured by time domain or frequency domain or both analysis of pulsatile signals from the skin from one or more of the cameras (R, G, B, I, other) in one or more colors. A standard location for measurement of heart rate is an area above the eyebrows on the forehead but other sites can be used to provide signal.

Respiratory Rate: Can be measured by time domain or frequency domain or both analysis of pulsatile signals from the skin from one or more of the cameras (R, G, B, I, other) in one or more colors. A standard location for measurement of respiratory rate is an area above the eyebrows on the forehead or the chest. Other areas can be used to provide the signal. Depth imaging of the chest can be used with or without or in combination with Eulerian magnification to provide input to the respiratory rate algorithm.

Tidal Volume: Dimensions of the chest can be obtained from the video image from RGB, IR, Depth, or stereoscopic cameras. Motion of the chest wall can be obtained from the depth or stereoscopic cameras. An algorithm that either explicitly calculates tidal volume from chest dimensions and chest wall motion can be used or an empiric model can be developed using statistical modelling, neural network, or deep learning algorithms. The model would be trained to produce tidal volume from single or multiple video inputs.

SpO2: Video signals from the RGB and IR camera(s) would be decomposed into individual colors. The peak and trough of each color channel would be identified. The ratios of (RedPeak/RedTrough)/(IRPeak/IRTrough), (GreenPeak/GreenTrough)/(IRPeak/IRTrough), (BluePeak/BlueTrough)/(IRPeak/IRTrough), (RedPeak/RedTrough)/(GreenPeak/GreenTrough), (RedPeak/RedTrough)/(BluePeak/BlueTrough), (GreenPeak/GreenTrough)/(BluePeak/BlueTrough), would be calculated. A calibration parameter would be adjusted for each individual color ratio. One or multiple ratios would be utilized based on signal quality, calibration, and lighting conditions.

Perfusion: The pulsatile color or IR signal will be calculated from the surface of the skin and mapped into a pulsatility image. The IR color signal can produce the image in the dark or through clothing or blankets. The level and gain of the displayed signal can be adjusted manually or automatically to produce the best pulsatility image. Areas of interest can be identified and highlighted such as a plastic surgery free flap or a limb with possible ischemia or compartment syndrome.

Systolic Blood Pressure: The pulsatile color or IR signal will be calculated from the surface of the skin. Signal averaging will be performed based on the peak or trough signal. A signal averaged pulsatile waveform will be produced. An explicit algorithm based on wave reflection or other approach can be produced to map the pulsatile signal to systolic blood pressure. In an alternative embodiment, machine learning (e.g., using a neural network model) can be used to create a predictive model from pulsatile signal to systolic blood pressure. In an alternative embodiment, a deep learning model will be produced mapping the video image in RGB or IR to systolic blood pressure with or without extracting parameters or doing prefiltering or signal averaging.

Diastolic Blood Pressure: The pulsatile color or IR signal will be calculated from the surface of the skin. Signal averaging will be performed based on the peak or trough signal. A signal averaged pulsatile waveform will be produced. An explicit algorithm based on wave reflection or other approach can be produced to map the pulsatile signal to diastolic blood pressure. In an alternative embodiment, machine learning (e.g., using a neural network model) can be used to create a predictive model from pulsatile signal to diastolic blood pressure. In an alternative embodiment, a deep learning model will be produced mapping the video image in RGB or IR to diastolic blood pressure with or without extracting parameters or doing prefiltering or signal averaging.

Mean Arterial Blood Pressure: The pulsatile color or IR signal will be calculated from the surface of the skin. Signal averaging will be performed based on the peak or trough signal. A signal averaged pulsatile waveform will be produced. An explicit algorithm based on wave reflection or other approach can be produced to map the pulsatile signal to mean arterial blood pressure. In an alternative embodiment, machine learning (e.g., using a neural network model) can be used to create a predictive model from pulsatile signal to mean arterial blood pressure. In an alternative embodiment, a deep learning model will be produced mapping the video image in RGB or IR to systolic blood pressure with or without extracting parameters or doing prefiltering or signal averaging.

Pain: Video signals in RGB, IR, or other colors will be recorded. A neural network or AI algorithm will identify the patient's face. Multiple locations will be identified on the patients face and facial action units identified. Facial action unit changes will then be mapped by neural network or other AI algorithm to pain measured either by VAS (Visual Analogue Scale) or Critical-Care Pain Observation Tool (CPOT), or both, or by using another pain measurement tool. In an alternative embodiment, the image of the patient's face in RGB, IR, or other color spectrum will be fed into a deep learning algorithm and matched to a pain score. In an alternative embodiment, images of the face, and or body, limbs, and or sounds, and or vital signs (e.g., HR and RR) will be fed into a deep learning or other statistical model or neural network trained to estimate the output of a reference pain measurement tool. The combination of vital signs and facial motions from action units and other signals may be analyzed by individual or multiple neural networks or other analytic tools to predict a pain score.

Pain measurement tools that may be used include but are not limited to:

Alder Hey Triage Pain Score, Behavioral Pain Scale (BPS), Brief Pain Inventory (BPI), Checklist of Nonverbal Pain Indicators (CNPI), Clinical Global Impression (CGI), COMFORT scale, Color Scale for Pain, Critical-Care Pain Observation Tool (CPOT), Dallas Pain Questionnaire, Descriptor differential scale (DDS), Dolorimeter Pain Index (DPI), Edmonton Symptom Assessment System, Face Legs Activity Cry Consolability scale, Faces Pain Scale-Revised (FPS-R), Global Pain Scale, Sequens algofunctional index: a composite measure of pain and disability, with separate self-report questionnaires for hip and knee OA (osteoarthritis), Mankoski Pain Scale, McGill Pain Questionnaire (MPQ), Multiple Pain Rating Scales, Neck Pain and Disability Scale-NPAD, Numerical 11 point box (BS-11), Numeric Rating Scale (NRS-11), Oswestry Disability Index, Palliative Care Outcome Scale (PCOS), Roland-Morris Back Pain Questionnaire, Support Team Assessment Schedule (STAS), Wharton Pain and Impairment Numeric Scale (Wharton PAIN Scale), Wong-Baker FACES Pain Rating Scale, Visual analog scale (VAS), Abbey pain scale for people with end-stage dementia, AUSCAN: Disease-Specific, to assess hand osteoarthritis outcomes., Colorado Behavioral Numerical Pain Scale (for sedated patients), CPOT For those who can't self report, Osteoarthritis Research Society International-Outcome Measures in Rheumatoid Arthritis Clinical Trials (OARSI-OMERACT) Initiative, New OA Pain Measure: Disease-Specific, Osteoarthritis Pain, Oucher Scale for Pediatrics, Pain Assessment in Advanced Dementia (PAINAD), Pediatric Pain Questionnaire (PPQ) for measuring pain in children, Premature Infant Pain Profile (PIPP) for measuring pain in premature infants, Schmidt Sting Pain Index and Starr sting pain scale both for insect stings, WOMAC: Disease-Specific, to assess knee osteoarthritis outcomes.

Delirium: A neural network, statistical model, or other algorithm will take video signals, and or sounds and map those images to a measurement of delirium. Patient limb motions, verbalizations, position in the bed, limb motions, eye movement, and other inputs may be used singularly or in combination to be analyzed by a neural network, deep learning network, statistical model, or other analytic tool to predict a delirium score. Additional data may or may not be added to the predictive model from the electronic health care record including, but not limited to, medications administered, past medical history, age, pre-existing medical conditions, and sedatives administered. Delirium will be match to a diagnosis of delirium and may or may not be measure with an objective tool including but not limited to: Richmond Agitation and Sedation Scale (RASS)—highly sensitive and specific for diagnosing delirium in older patients, Observational Scale of Level of Arousal (OSLA)—highly sensitive and specific for diagnosing delirium in older patients, Confusion Assessment Method (CAM), Delirium Observation

Screening Scale (DOS), Nursing Delirium Screening Scale (Nu-DESC), Recognizing Acute Delirium As part of your Routine (RADAR), 4AT (4 A's Test), Delirium Diagnostic Tool-Provisional (DDT-Pro), also for subsyndromal delirium.

Delirium Risk Factor Prediction: Risk factors for delirium will be assessed from the environment including sound levels by time of day, light levels by time of day, disturbances (e.g., sound, light, alarms, and clinical visits) during normal sleeping hours. The most important predisposing factors for delirium will be obtained from the medical record including: 1) 65 or more years of age, Male sex) Cognitive impairment/dementia; 2) physical comorbidity (biventricular failure, cancer, cerebrovascular disease); 3) psychiatric comorbidity (e.g., depression); 4) sensory impairment (vision, hearing); 5) functional dependence (e.g., requiring assistance for self-care or mobility); 6) dehydration/malnutrition; 7; drugs and drug-dependence, and 8) alcohol dependence.

Precipitating factors include, for example: 1) prolonged sleep deprivation; 2) environmental, physical/psychological stress (e.g., inadequately controlled pain, admission to an intensive care unit, immobilization, use of physical restraints, urinary retention, use of bladder catheter, emotional stress, and severe constipation/fecal impaction); 3) medications, such as sedatives (benzodiazepines, opioids), anticholinergics, dopaminergics, corticosteroids, polypharmacy; 4) General anesthetic; and 5) substance intoxication or withdrawal.

Primary neurologic diseases include, for example: 1) severe drop in blood pressure, relative to the patient's normal blood pressure (orthostatic hypotension) resulting in inadequate blood flow to the brain (cerebral hypoperfusion); 2) stroke/transient ischemic attack (TIA); 3) intracranial bleeding; and 4) meningitis and encephalitis.

Concurrent illness include, for example: 1) infections, especially respiratory (e.g., pneumonia, COVID-19) and urinary tract infections; 2) iatrogenic complications; 3) hypoxia, hypercapnea, and anemia; 4) poor nutritional status, dehydration, electrolyte imbalances, and hypoglycemia; 5) shock, heart attacks, and heart failure; 6) metabolic derangements (e.g., SIADH, Addison's disease, and hyperthyroidism); 7) chronic/terminal illness (e.g., cancer); 8) post-traumatic event (e.g., fall, fracture); and 9) mercury poisoning (e.g., Erethism).

Surgery includes, for example, cardiac, orthopedic, prolonged cardiopulmonary bypass, and thoracic surgeries.

A delirium risk score will be presented. Risk factors that can be reduced by changes in care plan such as reduced disturbances, light levels, sounds, medication choices will be identified.

Mood—Video signals in RGB, IR, or other colors will be recorded. A neural network or AI algorithm will identify the patient's face. Facial identification will be performed to ensure the correct patient is analyzed. Multiple locations will be identified on the patient's face and facial action units identified. Facial action unit changes will then be mapped by neural network or other AI algorithm to mood. Multiple parameters of mood will be displayed including but not limited to: anger, disgust, fear, happiness, sadness, surprise, depression, and neutral. Mood will be validated by patient report for the training session. Depression will be validated by PHQ-9 or other depression assessment tools.

Patient Call System: A two-way video and sound communication system is envisioned. Activation of the call system can be by voice command or hand motion or other inputs. Voice commands such as but not limited to “Nurse, can I have a . . . ” will activate the voice and speech recognition system and activate the two-way video and sound communication system. In an alternative embodiment, the voice command will be converted by speech to text message sent to clinical staff (nurse or doctor) or family. The two-way video and sound communication system can be used to communicate to family, friends, clinical staff, or anyone with a computer or smart phone.

Out of Bed Alarm: Video images of the patient's room will be obtained in RGB, IR, depth, or stereoscopic, or other color. Machine vision will be used to identify the patient's bed or other location. In an alternative embodiment, the area the patient is supposed to be located in can be identified by location on a computer screen with delimiters. Delimiters can be either automatically placed by the machine vision system or manually by clinical or other staff. The patient will then be identified in the bed or other area. If the patient's arms, legs, torso, or other identified area passes or begins to pass beyond the delimiter a notification will be flagged or sent or an alarm sounded depending on settings. Video imaging may or may not be sent to the central control station by settings.

Stroke: One of the most common types of stroke are strokes that affect the motor cortex. Patient motion of the limbs (arms and legs) and face will be tracked and time. Time for last motion of each limb will be recorded. Time for motion of the face will be recorded. Time for last intelligible speech command will be recorded. A setting in the system will be programmable of time to notify if an area of the body, which previously had motion, is no longer moving. Such as time since left arm last moved. Time since right arm last moved. Clinical staff can set the time for an alert, or it can be adjusted automatically to reduce false alarm rates. If a patient has a preexisting neurologic abnormality such as prior stroke, baseline mobility will be recorded and areas with normal motion tracked. If a patient has a preexisting dysarthria or is aphasic, the system will note the pre-existing condition and alarms will be set accordingly. The system will provide multiple factors, last identified movement of the suspected area, such as time left arm moved last. The system will identify the time since motion or function was normal. The system will have a recording of baseline motor and speech activity for reference. The system can be set to alert clinical staff to a change from baseline mobility after a specified time interval.

Regional Block: Local anesthetics affect nerves based on nerve size, myelination, and local anesthetic concentration. Because sympathetic nerves are small diameter fibers, they are rapidly and easily blocked by local anesthetic concentrations. Sympathetic nerve block effects on local blood flow can cause vasodilation. Vasodilation affects pulsatility imaging. The pulsatile image of areas of the body will be calculated. Variations in pulsatility imaging of the skin will be used to map the dermatomal level of the sympathetic block caused by the local anesthetic. Dermatomal maps and or levels will be presented for assessment of regional block.

Obstructive Breathing: Dimensions of the chest can be obtained from the video image from RGB, IR, Depth, or stereoscopic cameras. Motion of the chest wall can be obtained from the depth or stereoscopic cameras. An algorithm that either explicitly calculate tidal volume and identifies obstructive breathing from chest dimensions and chest wall motion can be used or an empiric model can be developed using statistical modelling, neural network, or deep learning algorithms. The model would be trained to identify obstructive breathing from single or multiple video inputs. Obstructive breathing can be identified by paradoxical motion of the chest wall and supraclavicular area. Paradoxical motion of the chest and or low tidal volumes will be identified. Dyscoordinated breathing patterns will be identified and warnings of possible residual nondepolarizing neuromuscular blockade will be identified by paradoxical or abnormal breathing and or motor activity patterns.

Vomiting: Dimensions of the chest can be obtained from the video image from RGB, IR, Depth, or stereoscopic cameras. Motion of the chest wall, head, and face, for example, can be obtained from the depth or stereoscopic cameras. An algorithm that either explicitly identifies vomiting from chest wall and head motion can be used or an empiric model can be developed using statistical modelling, neural network, or deep learning algorithms. The model would be trained to identify vomiting from single or multiple video inputs.

Shivering: Motion of the chest, limbs, muscles can be obtained from the video image from RGB, IR, Depth, or stereoscopic cameras. Eulerian magnification or other techniques can be used to identify rapid motions. Motion of the chest wall, head, face, and limbs, for example, can be obtained from the depth or stereoscopic cameras. An algorithm that either explicitly identifies shivering from patient motion can be used or an empiric model can be developed using statistical modelling, neural network, or deep learning algorithms. The model would be trained to identify shivering from single or multiple video inputs.

Congestive Heart Failure (CHF): Congestive heart failure presents a constellation of symptoms. Congestive heart failure (CHF), is a syndrome, a group of signs and symptoms caused by an impairment of the heart's blood pumping function. Symptoms typically include shortness of breath, excessive fatigue, and leg swelling. The shortness of breath may occur with exertion or while lying down, and may wake people up during the night. The severity of heart failure is measured by the severity of symptoms during exercise. Other conditions that may have symptoms similar to heart failure include obesity, kidney failure, liver disease, anemia, and thyroid disease. Common causes of heart failure include coronary artery disease, heart attack, high blood pressure, atrial fibrillation, valvular heart disease, excessive alcohol consumption, infection, and cardiomyopathy. These cause heart failure by altering the structure or the function of the heart or in some cases both. There are different types of heart failure: right-sided heart failure, which affects the right heart, left-sided heart failure, which affects the left heart, and biventricular heart failure, which affects both sides of the heart. Left-sided heart failure may be present with a reduced ejection fraction or with a preserved ejection fraction. Heart failure is not the same as cardiac arrest, in which blood flow stops completely due to the failure of the heart to pump effectively. Diagnosis is based on symptoms, physical findings, and echocardiography. Blood tests, and a chest x-ray may be useful to determine the underlying cause. CHF can be identified by images from the video system extracting a pattern of parameters including heart rate, respiratory rate, blood pressure, and perfusion. A multi-parameter model will be produced using neural networks, or statistical modeling, or deep learning from images and other parameters such as vital signs measured by AVD-M and or from the electronic health record. The CHF model will be trained using AI, neural networks, deep learning, statistical modeling, or other analytic techniques.

Heart Rate Variability (HRV): Can be measured by time domain or frequency domain or both. Analysis of pulsatile signals from the skin from one or more of the cameras (R, G, B, I, other) in one or more colors will be used. A standard location for measurement of heart rate variability is an area above the eyebrows on the forehead but other sites can be used to provide signal. The variation in the time between peaks or between troughs can be used to measure HRV. HRV is a predictor of sympathetic tone and may be used in other algorithms to predict outcomes.

Predictive Algorithms: There is significant morbidity and mortality from a respiratory and or cardiac arrest. Review of vital sign data prior to a cardiac arrest in hospital frequently identifies a slow and prolonged deterioration leading to the cardiac arrest. Some clinical events such as arrythmias, pulmonary emboli, or embolic stroke are sudden events that are difficult to predict. But many clinical events have a prolonged temporal pattern where if the problem is identified early, the progression of events leading to a possible respiratory or cardiac arrest can be stopped avoiding significant morbidity and mortality. To identify this progression of events leading to respiratory and cardiac arrest continuous or frequent monitoring of vital signs combined with analysis of the combination of the vital sign data is needed. The AVD-M system provides continuous measurement of multiple vital signs (e.g., HR, RR, SpO2, and BP). Statistical, neural network, deep learning, AI, or other multiparameter models based on data from the AVD-M system, and or electronic health care record (EHR) data will be used to predict the probability of respiratory or cardiac arrest, code blue, and or rapid response calls. A multidimensional model will provide a dimensionless parameter that predicts impending clinical events. Data from EHR records and or AVD-M data will be used to train the predictive model.

Psychiatric Monitoring: Patients in in-patient psychiatric wards need to be monitored on a continuous and or frequent basis. Remote, non-contact monitoring, where the lack of leads reduces the risk of suicide or suicide attempts would reduce risk. Infrared monitoring that does not disturb sleep will reduce risk, disturbances, and improve staff and patient safety. HR, RR, BP, temperature, mood, depression, activity will be monitored continuously with no sensors on the patients.

Cough: Dimensions of the chest can be obtained from the video image from RGB, IR, Depth, or stereoscopic cameras. Motion of the chest wall, head, and face, for example, can be obtained from the depth or stereoscopic cameras. An algorithm that either explicitly identifies and identifies cough from chest wall and head motion can be used or an empiric model can be developed using statistical modelling, neural network, or deep learning algorithms. The model would be trained to identify cough from single or multiple video inputs.

Temperature: Patient temperature can be measured from infrared imaging of the body. Distribution of patient skin temperature can be presented visually to show peripheral vasoconstriction or vasodilation as part of perfusion imaging, vasodilatory shock detection, and other clinical presentations.

Depression: Video signals in RGB, IR, or other colors will be recorded. A neural network or AI algorithm will identify the patient's face. Facial identification will be performed to ensure the correct patient is analyzed. Multiple locations will be identified on the patients face and facial action units identified. Facial action unit changes will then be mapped by neural network or other AI algorithm or other statistical model to mood. Multiple parameters of mood will be displayed including but not limited to: anger, disgust, fear, happiness, sadness, surprise, depression, and neutral. Mood will be validated by patient report for the training session. Psychomotor retardation, speech patterns, lack of speech, speech content, and body movements may be added to multiparameter models of depression. Depression will be validated by PHQ-9 or other depression assessment tools.

Skin blood flow: The pulsatile color or IR signal will be calculated from the surface of the skin and mapped into a pulsatility image. The IR color signal can produce the image in the dark or through clothing or blankets. The level and gain of the displayed signal can be adjusted manually or automatically to produce the best pulsatility image providing skin blood flow. Areas of interest can be identified and highlighted such as a plastic surgery free flap or a limb with possible ischemia or compartment syndrome.

Muscle blood flow: Muscle blood flow will be inferred from signals from the skin and deeper structures as skin blood flow reflects underlying muscle blood flow. The pulsatile color or IR signal will be calculated from the surface of the skin and mapped into a pulsatility image. The IR color signal can produce the image in the dark or through clothing or blankets. The level and gain of the displayed signal can be adjusted manually or automatically to produce the best pulsatility image providing skin and muscle blood flow. Areas of interest can be identified and highlighted such as a plastic surgery free flap or a limb with possible ischemia or compartment syndrome.

Shock: There are multiple types of shock including vasodilatory shock, cardiogenic shock, hypovolemic shock, anaphylactic shock, neurogenic shock, septic shock. Each type of shock has a variation in the clinical presentation. For example, vasodilatory shock has increased skin perfusion. Cardiogenic shock will have vasoconstriction with poor peripheral perfusion. Hypovolemic shock will have low blood pressure and vasoconstriction and a likely source of bleeding or volume loss. Anaphylactic shock may have cutaneous and or respiratory symptoms. Neurogenic shock will likely be vasodilatory and may have minimal muscle activity or motion. Septic shock may have the production of nitric oxide which increases the methemoglobin levels from the routine less than 0.3% to greater than 1.5%. Increases in methemoglobin levels will change skin tone towards grey or darker color in constellation with hypotension, poor perfusion, tachycardia, and increases in temperature. Methemoglobin (MetHb) is a dysfunctional form of hemoglobin that is incapable of transporting oxygen, thus reducing blood oxygenation, and potentially inducing tissue hypoxemia. Because, AVD-M monitoring has four colors (R, G, B, I) it can be calibrated to detect methemoglobin levels in patients. Combinations of parameters which may include or not include: HR, RR, BP, temperature, perfusion, temperature mapping, perfusion mapping, SpO2, hemoglobin levels will be used to detect and diagnose shock and different types of shock: vasodilatory shock, cardiogenic shock, hypovolemic shock, anaphylactic shock, neurogenic shock, septic shock. Statistical modeling, neural networks, deep learning, or empiric models, with or without additional information from the electronic health record will be used to identify and or diagnose shock and or type of shock.

Smart Alarms: The sensitivity and specificity of data from a monitoring system must be sufficient and the frequency of false alarms low enough that the information is clinically useful to staff. Smart alarm technology will be used that combines multiple parameters, checking of data, improvements in signal to noise, and artificial intelligence to reduce the incidence and frequency of false alarms while maintaining acceptable sensitivity and specificity. Since the AVD-M system is designed as a continuous early warning monitor where the results are checked by clinical evaluation by nurses and physicians in conjunction with standard contact monitoring, alarm limits can be adjusted to reduce false alarm rates. The low signal to noise ratio of camera based alarms can be compensated for by continuous monitoring, checking with other alarm parameters, and composite alarms. Rather than a simple, alarm if respiratory rate is less than 6 breaths a minute for 20 seconds, the clinical situation can be evaluated. If the patient is talking, then respirations can be assumed and the failure to detect 20 seconds of respiratory data can be ignored. Signal quality can be used to adjust vital sign reporting from a standard of “absolute accuracy” to “absolute less accurate” to “relative measurements”. For example, measurements such as pulse oximetry will be less accurate in darkened rooms and will likely not work in absolute darkness without an external light source. If the heart rate and respiratory rates indicate there may be a clinical problem lights can be turned on to get a pulse oximetry reading. If respiratory rate and heart rate done by infrared in the dark indicate normal vital signs but the pulse oximeter can read secondary to inadequate visible light, the pulse oximeter function will be suspended to avoid continuous alarms for poor signal strength or it may be measured periodically rather than continuously based on other vital signs and clinical needs.

Platform: The AVD-M system has camera(s) including, but not limited to, RGB, IR, depth, and stereoscopic in addition to microphone(s) or microphone arrays, speaker, monitor, and input and output devices (e.g., including lights of different color spectrum including, but not limited to, red, green, blue, white, and infrared). This monitoring platform has a computer, software, with or without a graphical processor unit (GPU), and networking capabilities. The hardware and software create a platform for medical monitoring that can provide input and output capabilities for other software utilizing the same platform. Natural language processing software, predictive models, models that combine data from the AVD-M sensors with data from the electronic health care record (EHR), and software that extracts other parameters or extract parameters with better accuracy, sensitivity, specificity, and lower false alarm rates, for example, may be hosted on the AVD-M platform. AVD-M software may also be run on patient's home computers utilizing the built in or external camera, microphones, and speakers to provide data for telemedicine visits or home health monitoring.

By extension, this or a similar system might also be used in the hospitals, nursing homes, operating rooms, recovery rooms, intensive care units, step down units, wards, hotels, home, schools, psychiatric facilities, airports, waiting rooms, transportation hubs, buses, planes, trains, or other public or private areas.

It is a further object to provide a simple method for monitoring people and providing information without physical contact with or sensors on the person. The system is capable of continuously monitoring people and providing multiple parameters including but not limited to: Heart rate, respiratory rate, oxygen saturation, Blood pressure, tidal volume, perfusion, pain, delirium, mood, patient call and communication, speech recognition, out of bed alarm, stroke, regional block level, obstructive breathing, vomiting, shivering, congestive heart failure, heart rate variability, predictive algorithms, cough, temperature, depression, skin blood flow, muscle blood flow, shock, and a platform for monitoring.

In addition to the foregoing, AVD-M monitoring can be used in transportation settings such as cars, planes, buses, trains, or other forms of transportation to assess function of the operator as well as presence of persons in the vehicle such as infants to avoid the hot car death scenario.

AVD-M monitoring can be used in a number of other situations including but not limited to: operators of dangerous machinery, subject's mobility (e.g., their gait in a nursing home or care facility), logistical issues in hospitals such as room turnover and room availability, stress level of passengers or crew (e.g., at security checkpoints), tiredness of staff in specific high risk environments such as transportation, systolic or pulse pressure variation as an indicator of volume status, fall detection in hospitals, nursing homes, home environment, communication in hospitals including incoming requests for a call may be answered (“picked up”) by the patient via audio and/or gesture, voice or gestures can be used by patient to interact with environment (e.g., turning light on/off, change TV channels, change volume of TV, the closing the window/blinds). Patient room automation, similar to home automation, can include monitoring of a patient's movements (in addition to facial expression monitoring) to estimate mood, and depression. AVD-M software, algorithms or components may be able to run on third-party hardware such as patient home computers during telemedicine or for family monitoring. Additional fields can include monitoring of firefighters and athletes, for example, to detect exhaustion levels, assess their ability to continue to perform, monitoring for triage in mass casualty events or battlefield situations, where a larger number of people need to be assessed quickly, combined with tele-photo lens monitoring of far away individuals, e.g., in law enforcement situations (hostage, hostage taker) or battlefield situations to establish signs of life to gauge need to recover, and in mass trauma events to triage or detect signs of life.

The AVD-M monitor provides a platform for monitoring utilizing a camera or cameras or camera array consisting of but not limited to a red, green, blue, infrared, depth, and or stereoscopic, or camera in other frequency ranges, and a microphone or microphone array, a computer, other input output devices, and or a graphical processor unit, and software either resident on the computer or remotely analyzing the input output data from the computer. The sensors detect the patient, identify the patient, locate areas of the body to be monitored, and calculate output parameters continuously or intermittently, and provide clinical information, alarms, and vital sign status of patients. The AVD-M system has networking capabilities to obtain and transmit information to the electronic health care record (EHR), and clinicians, families, and hospital administration as required.

A means for remote, non-contact monitoring of patients utilizing camera(s) and or camera arrays, microphones and or microphone arrays, speakers, monitors, networking, computer, and software to provide vital sign and other information for clinical care.

An apparatus for remote, non-contact monitoring of patients utilizing camera(s) and or camera arrays, microphones and or microphone arrays, speakers, monitors, networking, computer, and software to provide vital sign, logistical, and other information for clinical care.

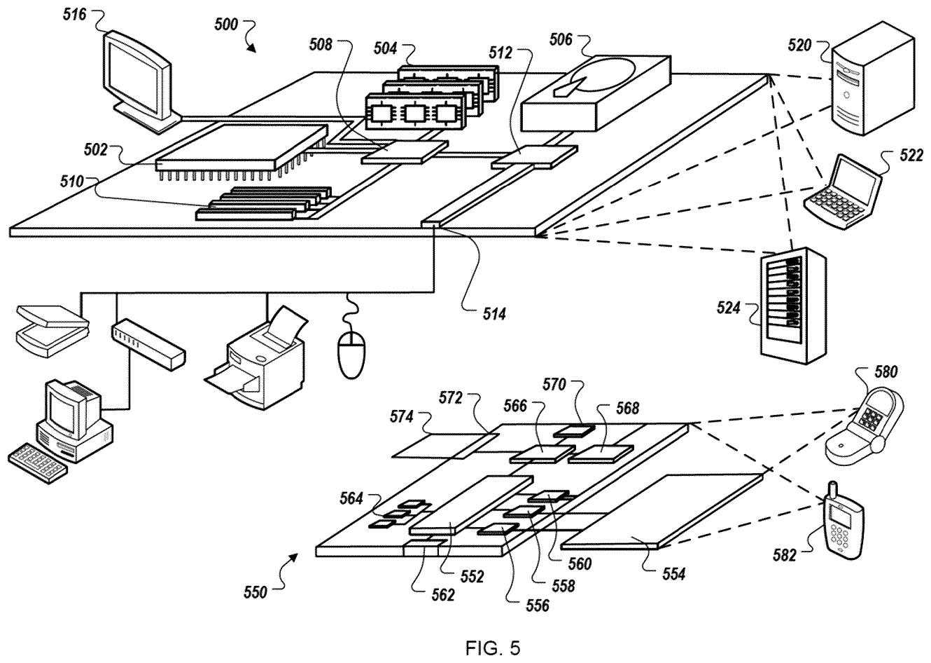

FIG. 5 shows an example of a computing device 500 and an example of a mobile computing device that can be used to implement the techniques described herein. The computing device 500 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 500 includes a processor 502, a memory 504, a

storage device 506, a high-speed interface 508 connecting to the memory 504 and multiple high-speed expansion ports 510, and a low-speed interface 512 connecting to a low-speed expansion port 514 and the storage device 506. Each of the processor 502, the memory 504, the storage device 506, the high-speed interface 508, the high-speed expansion ports 510, and the low-speed interface 512, are interconnected using various busses, and can be mounted on a common motherboard or in other manners as appropriate. The processor 502 can process instructions for execution within the computing device 500, including instructions stored in the memory 504 or on the storage device 506 to display graphical information for a GUI on an external input/output device, such as a display 516 coupled to the high-speed interface 508. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 504 stores information within the computing device 500. In some implementations, the memory 504 is a volatile memory unit or units. In some implementations, the memory 504 is a non-volatile memory unit or units. The memory 504 can also be another form of computer-readable medium, such as a magnetic or optical disk.

The storage device 506 is capable of providing mass storage for the computing device 500. In some implementations, the storage device 506 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer-or machine-readable medium, such as the memory 504, the storage device 506, or memory on the processor 502.

The high-speed interface 508 manages bandwidth-intensive operations for the computing device 500, while the low-speed interface 512 manages lower bandwidth-intensive operations. Such allocation of functions is exemplary only. In some implementations, the high-speed interface 508 is coupled to the memory 504, the display 516 (e.g., through a graphics processor or accelerator), and to the high-speed expansion ports 510, which can accept various expansion cards (not shown). In the implementation, the low-speed interface 512 is coupled to the storage device 506 and the low-speed expansion port 514. The low-speed expansion port 514, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 500 can be implemented in a number of different forms, as shown in the figure. For example, it can be implemented as a standard server 520, or multiple times in a group of such servers. In addition, it can be implemented in a personal computer such as a laptop computer 522. It can also be implemented as part of a rack server system 524. Alternatively, components from the computing device 500 can be combined with other components in a mobile device (not shown), such as a mobile computing device 550. Each of such devices can contain one or more of the computing device 500 and the mobile computing device 550, and an entire system can be made up of multiple computing devices communicating with each other.

The mobile computing device 550 includes a processor 552, a memory 564, an input/output device such as a display 554, a communication interface 566, and a transceiver 568, among other components. The mobile computing device 550 can also be provided with a storage device, such as a micro-drive or other device, to provide additional storage. Each of the processor 552, the memory 564, the display 554, the communication interface 566, and the transceiver 568, are interconnected using various buses, and several of the components can be mounted on a common motherboard or in other manners as appropriate.

The processor 552 can execute instructions within the mobile computing device 550, including instructions stored in the memory 564. The processor 552 can be implemented as a chipset of chips that include separate and multiple analog and digital processors. The processor 552 can provide, for example, for coordination of the other components of the mobile computing device 550, such as control of user interfaces, applications run by the mobile computing device 550, and wireless communication by the mobile computing device 550.

The processor 552 can communicate with a user through a control interface 558 and a display interface 556 coupled to the display 554. The display 554 can be, for example, a TFT (Thin-Film-Transistor Liquid Crystal Display) display or an OLED (Organic Light Emitting Diode) display, or other appropriate display technology. The display interface 556 can comprise appropriate circuitry for driving the display 554 to present graphical and other information to a user. The control interface 558 can receive commands from a user and convert them for submission to the processor 552. In addition, an external interface 562 can provide communication with the processor 552, so as to enable near area communication of the mobile computing device 550 with other devices. The external interface 562 can provide, for example, for wired communication in some implementations, or for wireless communication in other implementations, and multiple interfaces can also be used.

The memory 564 stores information within the mobile computing device 550. The memory 564 can be implemented as one or more of a computer-readable medium or media, a volatile memory unit or units, or a non-volatile memory unit or units. An expansion memory 574 can also be provided and connected to the mobile computing device 550 through an expansion interface 572, which can include, for example, a SIMM (Single In Line Memory Module) card interface. The expansion memory 574 can provide extra storage space for the mobile computing device 550, or can also store applications or other information for the mobile computing device 550. Specifically, the expansion memory 574 can include instructions to carry out or supplement the processes described above, and can include secure information also. Thus, for example, the expansion memory 574 can be provide as a security module for the mobile computing device 550, and can be programmed with instructions that permit secure use of the mobile computing device 550. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer-or machine-readable medium, such as the memory 564, the expansion memory 574, or memory on the processor 552. In some implementations, the computer program product can be received in a propagated signal, for example, over the transceiver 568 or the external interface 562.

The mobile computing device 550 can communicate wirelessly through the communication interface 566, which can include digital signal processing circuitry where necessary. The communication interface 566 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 568 using a radio-frequency. In addition, short-range communication can occur, such as using a Bluetooth, WiFi, or other such transceiver (not shown). In addition, a GPS (Global Positioning System) receiver module 570 can provide additional navigation-and location-related wireless data to the mobile computing device 550, which can be used as appropriate by applications running on the mobile computing device 550.

The mobile computing device 550 can also communicate audibly using an audio codec 560, which can receive spoken information from a user and convert it to usable digital information. The audio codec 560 can likewise generate audible sound for a user, such as through a speaker, e.g., in a handset of the mobile computing device 550. Such sound can include sound from voice telephone calls, can include recorded sound (e.g., voice messages and music files) and can also include sound generated by applications operating on the mobile computing device 550.

The mobile computing device 550 can be implemented in a number of different forms, as shown in the figure. For example, it can be implemented as a cellular telephone 580. It can also be implemented as part of a smart-phone 582, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a backend component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of the disclosed technology or of what may be claimed, but rather as descriptions of features that may be specific to particular embodiments of particular disclosed technologies. Certain features that are described in this specification in the context of separate embodiments can also be implemented in combination in a single embodiment in part or in whole. Conversely, various features that are described in the context of a single embodiment can also be implemented in multiple embodiments separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination. Similarly, while operations may be described in a particular order, this should not be understood as requiring that such operations be performed in the particular order or in sequential order, or that all operations be performed, to achieve desirable results. Particular embodiments of the subject matter have been described. Other embodiments are within the scope of the following claims.

What is claimed is:

1. A monitoring system, comprising:
one or more video systems configured to monitor a subject and capture vital information of the subject;
a computer client configured to i) receive the vital information from the one or more video systems, ii) determine, using at least the received vital information, at least one diagnosis of the subject, and iii) display the vital information and the at least one diagnosis;
a server in communication with the computer client and peripheral systems, including mobile devices, a central station, cloud-based storage, and an electronic health records (EHR) server, and configured to communicate with one or more of:
the central station configured to i) receive and display the vital information and the at least one diagnosis from the computer client, ii) receive inputs from a user of the central station, and iii) send, responsive to the received inputs, commands to the computer client;
the mobile devices configured to i) receive the vital information and the at least one diagnosis from the computer client, and ii) receive inputs from users of the mobile devices, and iii) send, responsive to the received inputs, commands to the computer client;
the cloud-based storage configured to store, in the cloud, and provide access to, cloud-formatted subject data generated from the vital information and the at least one diagnosis generated; and
the EHR server configured to store and provide access to EHR for the subject.

2. The monitoring system of claim 1:
wherein the vital information of the subject includes vital signs of a patient in a room of a healthcare facility,
wherein the one or more video systems include at least one camera in a camera array and at least one microphone in a patient's room configured to monitor the patient,
wherein the computer client is in the room in proximity to the subject and provides a display summarizing one ore more of: i) the vital signs of the patient, ii) the at least one diagnosis of the subject, and iii) a condition of the patient,
wherein the server is located in a ward of the healthcare facility that includes the room,
wherein the central station is a nurses' station in the ward of the healthcare facility,
wherein the mobile devices are carried by at least nurses in the ward, and
wherein communications are completed using one or more of LANs, WANs, and the Internet.

3. The monitoring system of claim 1:
wherein the vital information of the subject includes vital signs of a human being or an animal in a remote location;
wherein the one or more video systems include at least one camera and at least one microphone mounted on one or more of a drone, a fixed location, a vehicle, or a robotic device, and
wherein the computer client is nearby or distant from the subject and provides a display summarizing: i) the vital signs of the subject, and ii) at least one conclusion regarding a condition of the subject.

4. The monitoring system of claim 2, wherein capturing the vital information includes capturing a heart rate, a respiratory rate, a respiratory tidal volume, blood pressure, blood oxygenation, vomiting, shivering, congestive heart failure, heart rate variability, cough, temperature, skin blood flow, and muscle blood flow.

5. The monitoring system of claim 2, wherein determining the at least one diagnosis includes determining one or more of perfusion, pain, delirium, mood, probability of clinical deterioration, probability of cardiac, probability of respiratory arrest, depression, and shock.

6. The monitoring system of claim 2 further comprising a communication system comprising one-or two-directional video and sound communication between the subject and clinical staff or family, or others using a camera or a camera array and microphone or microphone array.

7. The monitoring system of claim 2 further comprising patient-controllable equipment configured to control, based on one or more of voice commands and gestures detected by the camera array, a television, a phone, lights, a call system, window shades, bed adjustments, or other devices.

8. The monitoring system of claim 2 wherein the computer client is further configured to:
monitor a position of the subject in bed and provide notifications to reduce risks of falling from the bed;
monitor for changes in subject movements of limbs, face, and body, determine, based on the monitoring, a time since a last movement of the subject, and changes from a baseline movement suggestive of a stroke or other neurologic injury;
monitor for changes in speech of the subject to identify a time since a last occurrence of normal speech and to monitor for changes from a baseline speech to identify one or more of a stroke, neurologic injury, aphasia, or dysarthria;
monitor skin perfusion to identify a level, a position, and an intensity of regional anesthesia block resulting from local anesthesia;
identify obstructive breathing or deconjugate breathing suggestive of sleep apnea, obstructive breathing, or residual non-depolarizing muscle block, or other issues interfering with normal ventilation;
monitor for vomiting by the subject;
monitor for shivering by the subject;
monitor for congestive heart failure; and
monitor heart rate variability.

9. The monitoring system of claim 2 wherein the computer client is further configured to predict a probability of clinical deterioration, cardiac, or respiratory arrest.

10. The monitoring system of claim 2 wherein the computer client is further configured to reduce risks in psychiatric facilities.

11. The monitoring system of claim 1 wherein determining the at least one diagnosis includes combining data derived from the camera array and microphone(s) with data from standard contact monitors of vital signs such as ECG, transmission or reflectance pulse oximetry, blood pressure measurement, temperature measurement and data from an electronic health care record to calibrate, check, validate, reduce false alarm rates, change sensitivities, and provide composite or other information based on the combination of signals.

12. The monitoring system of claim 3 wherein the computer client is further configured to establish signs of life in adverse conditions such as battlefield or disaster scenarios.

13. The monitoring system of claim 3 wherein the computer client is further configured to remotely evaluate vital signs of non-human animals in veterinary or farming applications.

14. The monitoring system of claim 1 wherein determining the at least one diagnosis includes using artificial intelligence, machine vision, data science, explicit, implicit, or learning algorithms to derive vital sign and other information from a camera, camera arrays, microphones, microphone arrays, or other sensors to provide clinical information.

15. A method comprising:
monitoring, using one or more video systems, a subject;
determining, based at least on the monitoring, vital health information of the subject; and
determining, based at least on the determined vital health information, at least one diagnosis of the subject.

16. The method of claim 15, further comprising:
displaying the vital health information and the at least one diagnosis.

17. The method of claim 15:
wherein the subject is a patient,
wherein the vital health information of the patient includes vital signs of the patient in a room of a healthcare facility, and
wherein the one or more video systems include at least one camera in a camera array and at least one microphone in a patient's room configured to monitor the patient.

18. The method of claim 15:
wherein the vital health information of the subject includes vital signs of a human being or an animal in a remote location; and
wherein the one or more video systems include at least one camera and at least one microphone mounted on one or more of a drone, a fixed location, a vehicle, or a robotic device.

19. The method of claim 17, further comprising:
monitoring a position of the subject in bed and provide notifications to reduce risks of falling from the bed;
monitoring for changes in subject movements of limbs, face, and body, determine, based on the monitoring, a time since a last movement of the subject, and changes from a baseline movement suggestive of a stroke or other neurologic injury;
monitoring for changes in speech of the subject to identify a time since a last occurrence of normal speech and to monitor for changes from a baseline speech to identify one or more of a stroke, neurologic injury, aphasia, or dysarthria;
monitoring skin perfusion to identify a level, a position, and an intensity of regional anesthesia block resulting from local anesthesia;
identifying obstructive breathing or deconjugate breathing suggestive of sleep apnea, obstructive breathing, or residual non-depolarizing muscle block, or other issues interfering with normal ventilation;
monitoring for vomiting by the subject;
monitoring for shivering by the subject;
monitoring for congestive heart failure; and
monitoring heart rate variability.

20. The method of claim 15, wherein the vital health information includes a heart rate, a respiratory rate, a respiratory tidal volume, blood pressure, blood oxygenation, vomiting, shivering, congestive heart failure, heart rate variability, cough, temperature, skin blood flow, and muscle blood flow.
